(19) United States
(12) Patent Application Publication
(10) Pub. No.: US 2007/0202156 A1
(43) Pub. Date: Aug. 30, 2007

(54) Adhesive preparation comprising sufentanil and methods of using same

(75) Inventors: Yuji Saeki, Miramar, FL (US); Masato Nishimura, Osaka (JP); Christopher L. Adams, Miramar, FL (US); Steven W. Sanders, Ft. Lauderdale, FL (US)

Correspondence Address: FOLEY AND LARDNER LLP;SUITE 500, 3000 K STREET NW, WASHINGTON, DC, 20007, US

(73) Assignee: Aveva Drug Deliverty Systems

(21) Appl. No.: 11/705,027

(22) Filed: Feb. 12, 2007

Related U.S. Application Data
(60) Provisional application No. 60/772,522, filed on Feb. 13, 2006.

(51) Int. Cl. A61K 9/70

(52) U.S. Cl. 424/449

(57) ABSTRACT

The present invention provides percutaneously absorbable adhesive preparations and transdermal compositions comprising Sufentanil, which are obtained from economic starting materials, have a constitution simpler than that of conventional Sufentanil adhesive preparations, and have effective skin permeability. The preparations and compositions comprise two polyisobutylenes having different molecular weights, a tackifier, a permeation enhancer.

### RELATED APPLICATION

This application claims the benefit of priority from U.S. Provisional Application No. 60/772,522, filed Feb. 13, 2006, the contents of which are incorporated herein by reference.

### FIELD OF THE INVENTION

The present invention provides a Sufentanil-comprising adhesive preparation or transdermal composition for continuous administration of Sufentanil through the skin.

### BACKGROUND

Narcotic analgesics have been widely used for the management of chronic pain, such as cancer pain, and have demonstrated great utility and therapeutic effect. Only a few narcotic analgesics have been provided as percutaneously absorbable adhesive preparations for treating chronic pain. Such dosage forms are desirable and contribute to the society as a new treatment method since percutaneously absorbable preparations have advantages over other administration routes.

However, the effect of narcotic analgesic can vary between individual subjects, and dose-related adverse events associated with drug administration can be problematic. Therefore, to achieve greater control and convenience of administration, percutaneously absorbable preparations have been used in the management of chronic pain.

In general, Sufentanil is more effective and has been associated with reduced adverse effects when compared to certain other narcotic analgesics, such as Morphine. Percutaneously absorbable preparations with Sufentanil having various constitutions have been considered and disclosed (U.S. Pat. Nos. 4,588,580; 4,806,341; 4,822,802; 4,927,408; and 5,656,285; 6,074,665). However, no percutaneously absorbable preparation comprising Sufentanil has been granted regulatory approval. The Sufentanil preparations in the aforementioned patents have complicated constitutions, are very expensive to produce, and are economically disadvantageous. To lower the cost, even if only slightly, it is necessary to simplify the composition to lower the step cost and utilize economical materials.

Additionally, since Sufentanil is a potent narcotic analgesic, there is a need for a delivery system, such as a transdermal composition, that provides for both safe as well as effective drug delivery to the systemic circulation. Thus, there is a need for a transdermal composition that can accomplish a relatively rapid, but controlled, onset of analgesia during the initial absorption time period and sustained delivery with unique features that provide continued analgesia, avoiding dose dumping or extreme variations in drug delivery. There also is a need for a transdermal composition that allows rapid discontinuation of treatment by removal of the system and subsequent rapid decline in skin absorption.

### SUMMARY

In one embodiment, a transdermal composition comprises a support and an adhesive layer laminated on one surface of the support, wherein the adhesive layer comprises a subsaturated amount of Sufentanil, a polyisobutylene blend comprising a first polyisobutylene and a second polyisobutylene having different viscosity average molecular weights, a tackifier, and a permeation enhancer compatible with the polyisobutylene blend and the tackifier. In one embodiment, the first polyisobutylene has a viscosity average molecular weight of about 600000 to about 1500000 and the second polyisobutylene has a viscosity average molecular weight of about 40000 to about 85000.

In another embodiment, the transdermal composition comprises an amount of Sufentanil effective to provide therapeutic levels for about 1 day, about 2 days, about 3 days, about 4 days, about 5 days, about 6 days, or about one week. In another embodiment, the transdermal composition comprises from about 0.5 mg to about 15 mg of Sufentanil. In a further embodiment, the transdermal composition comprises an amount of Sufentanil of about 0.5 mg, about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, or about 15 mg.

In another embodiment, the transdermal composition comprises a rate controlling membrane.

In another embodiment, the support comprises or consists of a non-porous film laminated to a porous film. In a further embodiment, the support comprises or consists of woven fabric, nonwoven fabric, knitted fabric, paper, a mechanically perforated sheet, or a combination of two or more thereof. In another embodiment, the support is translucent or transparent.

Another embodiment is directed to methods of treating a subject in need of pain relief, comprising applying to the skin of the subject a first Sufentanil transdermal composition comprising a support and an adhesive layer laminated on one surface of the support, wherein the adhesive layer comprises a subsaturated amount of Sufentanil, a polyisobutylene blend comprising a first polyisobutylene and a second polyisobutylene having different viscosity average molecular weights, a tackifier, and a permeation enhancer compatible with the polyisobutylene blend and the tackifier. In a further embodiment, the method provides therapeutic levels of Sufentanil in the subject for a period of time selected from the group consisting of about 1 day, about 2 days, about 3 days, about 4 days, about 5 days, about 6 days, and about one week.

In another embodiment, the method of treating a subject in need of pain relief further comprises applying to the skin of the subject a second Sufentanil transdermal composition comprising (a) a support; and (b) an adhesive layer laminated on one surface of the support, wherein the adhesive layer comprises a Sufentanil, a polyisobutylene blend comprising a first polyisobutylene and a second polyisobutylene having different molecular weights, a tackifier, and a permeation enhancer compatible with said two kinds of polyisobutylene and the tackifier. In a further embodiment, the method further comprises removing the first Sufentanil transdermal composition from the skin of the subject prior to applying the second Sufentanil transdermal composition.

In another embodiment, the method of treating a subject in need of pain relief achieves a plasma concentration of Sufentanil in the subject of less than about 2 pg/ml for a time period of about 30 minutes to about 2 hours after initial application of the Sufentanil transdermal composition. In another embodiment, the method achieves a plasma concentration of Sufentanil in the subject of greater than about 2 pg/ml within six hours after initial application of the Sufentanil transdermal composition. In another embodiment, the method reaches a peak plasma concentration of Sufentanil in the subject at about 12 hours to about 72 hours after initial application of the Sufentanil transdermal composition. In a further embodiment, the method reaches a peak plasma concentration of Sufentanil in the subject at about 24 hours after initial application of the Sufentanil transdermal composition. In a further embodiment, the method achieves a peak plasma concentration of Sufentanil in the subject of from about 1 to about 30 pg/ml per cm² of the composition.

In another embodiment, the Sufentanil transdermal composition comprises an amount of Sufentanil effective to deliver from about 0.5 to about 2 μg/hr Sufentanil per cm² of the composition over a duration of application of from at least about 1 day to about 7 days. In another embodiment, the Sufentanil transdermal composition comprises an amount of Sufentanil effective to maintain a Sufentanil delivery rate of at least about 1 μg/hr Sufentanil per cm² of the composition for a duration of application of from at least about 1 day to about 7 days. In a further embodiment, the duration is a period of time of about 1 day, about 2 days, about 3 days, about 4 days, about 5 days, about 6 days, and about one week.

In another embodiment, the Sufentanil transdermal composition comprises an amount of Sufentanil effective to deliver at least 1 μg/hr Sufentanil per cm² of the composition in the first 24 hours after initial application. In a further embodiment, the Sufentanil transdermal composition comprises an amount of Sufentanil effective to achieve, after the first 24 hours after initial application, a plasma concentration of Sufentanil in the subject of at least 1 pg/ml per cm² of the composition.

Both the foregoing general description and the following detailed description are exemplary and explanatory and are intended to provide further explanation of the invention as claimed. Other objects, advantages, and novel features will be readily apparent to those skilled in the art from the following brief description of the drawings and the detailed description of the invention.

### BRIEF DESCRIPTION OF THE DRAWINGS

FIG. 1 is a factor effect figure reflecting the analysis results of Experimental Example 2, which shows the SN ratio fluctuation (Table 7) at levels 1 to 2, or 1 to 3 of factors A-G (e.g., molecular weight, mixing ratio, concentration).

FIG. 2 and 3 are graphs reflecting the analysis results of Experimental Example 3, which show in vitro percutaneous flux of Sufentanil through human epidermis obtained from cryopreserved cadaver skin.

FIG. 4 is a graph reflecting the results of Experimental Example 4, which shows blood Sufentanil concentrations following percutaneous application of 2.7 cm² of adhesive preparation (transdermal composition) for 3 days.

FIG. 5 is a graph reflecting the analysis results of Experimental Example 5, which shows in vitro percutaneous flux of Sufentanil through human epidermis obtained from cryopreserved cadaver skin.

FIG. 6 is a graph reflecting the results of Experimental Example 6, which shows blood Sufentanil concentrations over 11 days during and following percutaneous application for 7 days of 2.7 cm² of adhesive preparation (transdermal composition).

### DETAILED DESCRIPTION

Described herein is a Sufentanil-comprising percutaneously absorbable adhesive preparation (e.g., transdermal composition) prepared from economical materials, which has a simpler composition or construction as compared to conventional preparations, is made by simple steps, and has effective functions of skin permeability and the like. Transdermal compositions described herein achieve controlled, yet relatively rapid, delivery of the drug for safe and effective treatment. For example, one embodiment provides a percutaneously absorbable adhesive preparation, or transdermal device or transdermal composition, comprising a support and an adhesive layer laminated on one surface of the support wherein the adhesive layer comprises a Sufentanil, two kinds of polyisobutylene having different molecular weights, a tackifier, and an organic liquid (e.g., permeation enhancer) compatible with the aforementioned two kinds of polyisobutylene and the aforementioned tackifier. While not wanting to be bound by any theory, it is believed that the use of the aforementioned polyisobutylenes, tackifier, and permeation enhancer offers an advantage of being able to control skin permeation by changing the mixing ratios of these components. Drug delivery also can be controlled via a rate controlling membrane, which is an optional component of the transdermal compositions described herein.

Also described herein are transdermally absorptive preparations (transdermal compositions) that are free of precipitated Sufentanil crystals, due at least in part to the high solubility of Sufentanil in the permeation enhancer, which is increased as compared to the solubility of Sufentanil in other preparations. In one embodiment, the transdermal composition comprises an adhesive layer comprising a subsaturated amount of Sufentanil. By a “subsaturated amount of Sufentanil” is meant a concentration of Sufentanil that is below its solubility or saturation point in the adhesive layer. A further advantage of such preparations is improved appearance, e.g., transdermal compositions described herein can have an aesthetically pleasing appearance.

According to the present invention, therefore, a preparation having effective function as compared to conventional preparations can be produced economically, whereby a fine Sufentanil preparation can be provided at a low cost.

As used herein, the singular forms “a,” “an,” and “the” designate both the singular and the plural, unless expressly stated to designate the singular only.

As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular term.

As used herein “subject” denotes any subject in need of pain therapy, including human subjects.

As used herein, the phrases “therapeutically effective amount” and “therapeutic level” mean that drug dosage or plasma concentration in a subject that provides the specific pharmacological response for which the drug is administered in a subject in need of such treatment. It is emphasized that a therapeutically effective amount or therapeutic level of a drug that is administered to a particular subject in a particular instance will not always be effective in treating the conditions/diseases described herein, even though such dosage is deemed to be a therapeutically effective amount by those of skill in the art. For convenience only, exemplary dosages, drug delivery amounts, therapeutically effective amounts and therapeutic levels are provided below with reference to adult human patients. Those skilled in the art can adjust such amounts in accordance with standard practices.

Technical and scientific terms used herein have the meanings commonly understood by one of ordinary skill in the art to which the present invention pertains, unless otherwise defined. Reference is made herein to various methodologies known to those of ordinary skill in the art. Publications and other materials setting forth such known methodologies to which reference is made are incorporated herein by reference in their entireties as though set forth in full. Any suitable materials and/or methods known to those of ordinary skill in the art can be utilized in carrying out the present invention. However, specific materials and methods are described. Materials, reagents and the like to which reference is made in the following description and examples are obtainable from commercial sources, unless otherwise noted.

The Sufentanil preparations or transdermal devices or transdermal compositions described herein are superior to prior narcotic transdermal dosage forms, as they enable the use of lower doses of Sufentanil. Sufentanil is related to fentanyl, which is a potent Schedule II opioid agonist. Schedule II opioid substances include fentanyl, hydromorphone, methadone, morphine, oxycodone, and oxymorphone, and have the highest potential for abuse and associated risk of fatal overdose due to respiratory depression. Lower doses of narcotic analgesics, such as Sufentanil, are preferable, as narcotic analgesics can become addictive and can be abused. Moreover, narcotic analgesic dosage forms having higher drug dosages may be a particular target for abuse and diversion. Thus, lower doses of Sufentanil minimize the possibility of both addiction and abuse and are therefore preferred.

Additional benefits associated with the therapeutic use of Sufentanil surround the emerging understanding of the genetic variation in the opiate receptor and its effects on individual patient response to analgesic agents. Although the general pharmacological mechanism of action remains consistent from patient to patient, minor differences in structure of the compounds and relative binding to the multiple genetic-determined characteristics of the opiate receptors, lead to individual differences in response to various narcotic analgesics. For this reason, multiple alternatives will benefit overall patient treatment by allowing individualization of treatment based on analgesic response. In addition, adaptive changes in receptor response may occur over time leading to the potential for continued patient response to an alternate analgesic when response declines due to the development of drug tolerance.

Sufentanil is a Fentanyl analogue. Fentanyl and Sufentanil have the following structures:

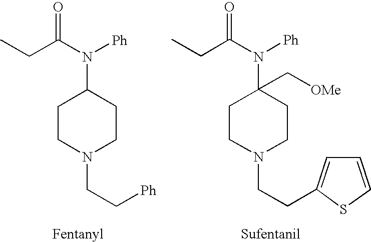

Like other opioid analgesics, Fentanyl and Sufentanil bind with stereospecific receptors at many sites within the central nervous system to alter processes affecting both the perception of and emotional response to pain. Although both Fentanyl and Sufentanil have good skin permeation properties, Sufentanil provides a more potent analgesic effect, allowing for the use of smaller amounts of drug to produce a therapeutic effect. Sufentanil has been reported to have a potency that is approximately 7 to 10 times that of Fentanyl.

One characteristic of Sufentanil is its extremely high octanol:water partition coefficient of 1757, which results in virtually instantaneous central nervous systems effects following intravenous administration. The high corresponding lipid solubility (log p=3.95) would be expected to translate into extremely rapid transcutaneous permeation leading to safety concerns for patients following application of a Sufentanil transdermal composition. However, it has been found that the transdermal compositions described herein exhibit a lag time to peak plasma concentration, thus providing a margin of safety that would not have been predicted. While not wanting to be bound by any theory, it is believed that a small amount of Sufentanil may be loaded into the skin lipid layer under the composition surface, which may explain the short, but useful, lag time for absorption.

The typical detection threshold for Sufentanil is about 2 pg/ml, It has been found that, after application of the transdermal compositions described herein to human subjects, Sufentanil is not detected in the blood, for about 30 minutes to about two hours after initial application, such as for 30 minutes to two hours after initial application, and that Sufentanil may not be detected in the blood for up to about six hours after initial application, such as up to six hours after initial application. Thus, for example, the plasma concentration of Sufentanil in a subject may reach greater than about 2 pg/ml, such as greater than 2 pg/ml, within six hours after initial application of the Sufentanil transdermal composition. It also has been found that peak plasma concentrations (Cₘₐₓ) may be achieved within from about 12 to about 72 hours (Tₘₐₓ), including about 24 hours, after initial application of the transdermal compositions described herein, such as within from 12 to 72 hours, or 24 hours, after initial application. Thus, the transdermal compositions are suitable for administration to patients requiring maintenance of analgesia beyond a few hours, including patients with pain lasting longer than one day.

The percutaneously absorbable adhesive preparations, or transdermal devices or transdermal compositions, described herein comprise a support and an adhesive layer comprising a subsaturated amount of Sufentanil, two kinds of polyisobutylene having different molecular weights, a tackifier, and a permeation enhancer compatible with the aforementioned two kinds of polyisobutylene having different molecular weights and the aforementioned tackifier, laminated on one surface of the support. While the preparation or transdermal composition can be produced conveniently from common and economical materials and with a simplified process, it shows superior properties (e.g., effective skin permeability, ability to control skin permeability and the like) as compared to those of the conventional preparations.

Transdermal Compositions

In accordance with one embodiment, the transdermal composition comprises a support and an adhesive layer laminated on one surface of the support, wherein the adhesive layer comprises a Sufentanil, a polyisobutylene blend comprising a first polyisobutylene and a second polyisobutylene having different molecular weights, a tackifier, and a permeation enhancer compatible with said polyisobutylene blend and said tackifier.

The form of the percutaneously absorbable adhesive preparation or transdermal composition is not particularly limited and may be, for example, a tape, a sheet, and the like. Suitable forms of transdermal compositions are known in the art.

The transdermal composition may be provided with a surface area of any suitable size, including from about 1 to about 30 cm², including about 2, about 5, about 10, about 15, about 20, or about 25 cm² including 1, 2, 5, 10, 15, 20, or 25 cm². In some embodiments, the size and shape of the transdermal composition are selected to suit the particular dosage requirements and/or aesthetic preferences of a given subject.

1. Adhesive Layer

A. Sufentanil

As noted above, the adhesive layer of the transdermal compositions described herein comprises Sufentanil.

In one embodiment, the adhesive layer comprises a subsaturated amount of Sufentanil, e.g., an amount of Sufentanil that is below the solubility or saturation point of Sufentanil in the adhesive layer. In some embodiments, the proportion of Sufentanil relative to the total weight of the adhesive layer is about 1.5 to about 5.0%, including about 1.7% to about 4.0%, such as 1.5% to 5.0%, or 1.7% to 4.0%. Such amounts may provide an advantageous balance of efficacy and cost considerations. When the proportion of Sufentanil is less than about 1.5%, a sufficient clinical effect may not be afforded, and when it exceeds about 5.0%, crystal precipitation may occur, which is economically and therapeutically disadvantageous.

While the appropriate dose of Sufentanil in the percutaneously absorbable adhesive preparation of the present invention (e.g., transdermal composition) typically varies depending on the age, body weight, symptoms etc. of a subject, a typical preparation or composition may comprise about 0.5 mg to about 15 mg of Sufentanil, such as 0.5 mg to 15 mg, and is generally applied to about 1 cm² to about 30 cm², including about 2 cm² to about 20 cm², of the skin of an adult human, using, for example, a transdermal composition with a surface area of about 1 cm² to about 30 cm², including about 2 cm²to about 20 cm², such as 1 cm²to 30 cm², or 2 cm² to 20 cm². In specific embodiments, the Sufentanil preparation (transdermal composition) comprises about 0.5 mg, about 1 mg, about 2 mg, about 3 mg, about 4 mg, about 5 mg, about 6 mg, about 7 mg, about 8 mg, about 9 mg, about 10 mg, about 11, mg, about 12 mg, about 13 mg, about 14 mg, or about 15 mg of Sufentanil, including 0.5 mg, 1 mg, 2 mg, 3 mg, 4 mg, 5 mg, 6 mg, 7 mg, 8 mg, 9 mg, 10 mg, 11 mg, 12 mg, 13 mg, 14 mg, or 15 mg.

B. Polyisobutylene Blend

As noted above, the adhesive layer of the transdermal compositions described herein contains two kinds of polyisobutylene having different molecular weights, e.g., a polyisobutylene blend comprising a first polyisobutylene and a second polyisobutylene having different molecular weights. The term “two kinds of polyisobutylene having different molecular weights” refers to a composition comprising a first polyisobutylene generally having one average molecular weight and a second polyisobutylene generally having a different average molecular weight than the aforementioned first polyisobutylene, where “average molecular weight” refers to viscosity average molecular weight.”

Viscosity average molecular weight can be determined by calculating a Staudinger-Index (J₀) by the Schulz-Blaschke equation from the flow time of capillary 1 of a Ubellohde's viscometer at 20° C. and from the following formula using the J₀ value:
J₀=ηₛₚ/c(1+0.31ηₛₚ)cm³/g (Schulz-Blaschke equation)
ηₛₚ=t/t₀−1
t: flow time of solution (Hagenbach-couette correction)
t₀: flow time of solution (Hagenbach-couette correction)
c: concentration of solution (g/cm³)
   J₀=3.06×10⁻² M̅v̅^{0.65}
M̅v̅: viscosity average molecular weight

The two kinds of polyisobutylene having different molecular weights are not particularly limited, and suitable polyisobutylenes are known in the art. In some embodiments, which may achieve fine adhesiveness and releaseability of Sufentanil, the first polyisobutylene has a viscosity average molecular weight of about 600,000 to about 1,500,000, including about 700,000 to about 1,350,000, and the second polyisobutylene has a smaller viscosity average molecular weight than that of the first polyisobutylene, including a viscosity average molecular weight of about 40,000 to about 85,000, or about 45,000 to about 65,000. Thus, in one embodiment, the first polyisobutylene has a viscosity average molecular weight of 600,000 to 1,500,000, including 700,000 to 1,350,000, and the second polyisobutylene has a viscosity average molecular weight of 40,000 to 85,000, or 45,000 to 65,000. Here, when the first polyisobutylene has a viscosity average molecular weight of less than about 600,000, the inner cohesion necessary for an adhesive layer tends to be unattainable, and when it exceeds about 1,500,000, the skin adhesion and tack of the adhesive layer tend to be degraded. Furthermore, when the second polyisobutylene has a viscosity average molecular weight of less than about 40,000, the adhesive layer becomes sticky and may possibly contaminate the skin surface, and when it exceeds about 85,000, the skin adhesion and tack of the adhesive layer tend to be degraded. Suitable polyisobutylenes can be supplied stably at economical costs from commercial sources.

In some embodiments, a mixing ratio of the above-described first polyisobutylene:second polyisobutylene is about 1:1.2 to about 1:2, including about 1:1.3 to about 1:1.8, by weight of polyisobutylenes. In one embodiment, a mixing ratio of the first and second polyisobutylenes is 1:1.2 to 1:2, including 1:1.3 to 1:1.8, by weight of polyisobutylenes. When the mixing ratio of the second polyisobutylene relative to the first polyisobutylene is less than about 1.2 by weight, the adhesive layer shows markedly decreased skin adhesion, and when the ratio exceeds about 2, the adhesive layer shows markedly decreased inner cohesion.

In some embodiments, the proportion of the two kinds of polyisobutylene having different molecular weights relative to the total weight of the adhesive layer is about 50% to about 83%, including about 55% to about 78%, based on the total weight of the adhesive layer. In one embodiment, the proportion of the two kinds of polyisobutylene is 50% to 83%, including 55% to 78%, based on the total weight of the adhesive layer. In some embodiments, the proportion of the first polyisobutylene (having a higher viscosity average molecular weight than that of the second polyisobutylene) of the two kinds of polyisobutylene having different molecular weights, relative to the whole weight of the adhesive layer, is about 18% to about 36%, including about 22% to about 32%. In one embodiment, the proportion of the first polyisobutylene relative to the whole weight of the adhesive layer is 18% to 36%, including 22% to 32%. In some embodiments, the proportion of the second polyisobutylene (having a smaller viscosity average molecular weight than that of the first polyisobutylene), relative to the total weight of the adhesive layer, is about 33% to about 48%, including about 35% to about 44%. In one embodiment, the proportion of the second polyisobutylene relative to the whole weight of the adhesive layer is 33% to 48%, including 35% to 44%.

C. Tackifier

As noted above, the transdermal compositions described herein comprise a tackifier. The tackifier used in the transdermal compositions may be appropriately selected from those known in the field of adhesive preparations. The tackifier can be, for example, a polybutene, rosin resin, terpene resin, petroleum resin, chroman resin, and the like. From the aspects of cost and fine tack, the following compounds are suitable: (i) a polybutene having a number average molecular weight of about 900 to about 2900, including about 980 to about 2000, or 900 to 2900, including 980 to 2000 (e.g., 1-butene polymer, 2-butene polymer, copolymer of 1-butene and 2-butene); and (ii) an alicyclic saturated hydrocarbon resin having a number average molecular weight of about 500 to about 860, including about 570 to about 710, including 500 to 800, including 570 to 710. When the number average molecular weight of polybutene of the above-mentioned (i) is less than about 900, the adhesive layer tends to become sticky, thus possibly posing problems in terms of handling property, and when it exceeds about 2900, the effect of the tackifier is difficult to show, and the tack of the adhesive layer tends to decrease. When the number average molecular weight of the alicyclic saturated hydrocarbon resin of the above-mentioned (ii) is less than about 500, the adhesive layer tends to be sticky, and when it exceeds about 860, compatibility becomes inferior. Examples of the alicyclic saturated hydrocarbon resin of the above-mentioned (ii) include, but are not limited to, commercially available hydrogenation petroleum resins manufactured by Arakawa Chemical Industries, Ltd., such as ARCON P-70, ARCON P-900, ARCON P-100 etc., and the like.

The number average molecular weight can be measured by the gel permeation chromatography (GPC) method or the vapor pressure osmometry (V.P.O.) method.

In some embodiments, the tackifier component of the transdermal composition comprises a combination of one or more kinds of the above-described tackifiers.

In some embodiments, the proportion of the tackifier relative to the total weight of the adhesive layer is about 15% to about 30%, including about 16% to about 28%. In some embodiments, the proportion of the tackifier relative to the total weight of the adhesive layer is 15% to 30%, including 16% to 28%. When the proportion of the tackifier is less than about 15%, the tack may be poor, and when it exceeds about 30%, the adhesive layer may undesirably show a propensity toward destruction.

D. Permeation Enhancer

As noted above, the transdermal compositions described herein comprise a permeation enhancer (organic liquid). The permeation enhancer used in the present invention is not particularly limited as long as it is compatible with the above-mentioned two kinds of polyisobutylene having different viscosity average molecular weights and the above-mentioned tackifier.

Examples of useful permeation enhancers include, but are not limited to, a fatty acid alkyl ester, a branched long-chain alcohol, and the like. Exemplary fatty acid alkyl esters include, but are not limited to, a fatty acid alkyl ester comprising a higher fatty acid having about 12 to about 16, including about 12 to about 14, carbon atoms, and a lower monovalent alcohol having about 1 to about 4 carbon atoms, including higher fatty acid havings 12 to 16, including 12 to 14, carbon atoms, and lower monovalent alcohols having 1 to 4 carbon atoms. As the above-mentioned higher fatty acid, suitable examples are lauric acid (C12), myristic acid (C14) and palmitic acid (C16). In some embodiments, myristic acid is used. Exemplary lower monovalent alcohols include, but are not limited to, straight chain and branched chain alcohols having about 1 to about 4 carbon atoms, including 1-4 carbon atoms. Specific examples include methyl alcohol, ethyl alcohol, propyl alcohol, isopropyl alcohol, butyl alcohol and the like. In some embodiments, isopropyl alcohol is used. In some specific embodiments, the preferable fatty acid alkyl ester used is isopropyl myristate. Exemplary branched long-chain alcohols include, but are not limited to, saturated or unsaturated branched long-chain alcohols having about 16 to about 22, including about 18 to about 20, carbon atoms, including those having 16-22, or 18-20, carbon atoms. Specific examples include isostearyl alcohol, octyldodecanol and the like. In some embodiments, isostearyl alcohol and/or octyldodecanol is used. In specific embodiments using a long-chain alcohol as the permeation enhancer, a branched long-chain alcohol is used. Non-branched long-chain alcohols may sometimes have poor compatibility with polyisobutylene.

In some embodiments, the permeation enhancer of the transdermal composition comprises a combination of one or more kinds of the above-described permeation enhancers.

Suitable organic liquids for use as permeation enhancers include, but are not limited to, a fatty acid alkyl ester and a branched long-chain alcohol. In some embodiments, isopropyl myristate, isostearyl alcohol and/or octyldodecanol are used. These embodiments may exhibit advantageous Sufentanil absorption enhancing effects, which may be due at least in part to the increased solubility of Sufentanil adhesive layers comprising such permeation enhancers.

In some embodiments, the permeation enhancer comprises a combination of a fatty acid alkyl ester and a branched long-chain alcohol. Such embodiments may have advantageous properties due to the solubility of Sufentanil in such organic liquids, also exhibiting enhanced skin permeation properties, and the good skin adhesiveness of such an adhesive layer. In specific embodiments, the mixing ratio of fatty acid alkyl ester and branched long-chain alcohol may be about 1:0.2 to about 1:5, including about 1:0.3 to about 1:4, by weight of these two organic liquid components. In one embodiment, the mixing ratio of fatty acid alkyl ester and branched long-chain alcohol is 1:0.2 to 1:5, including 1:0.3 to 1:4, by weight of these two organic liquid components. When the mixing ratio (of branched long-chain alcohol:fatty acid alkyl ester) by weight is less than about 0.2, the solubility of Sufentanil may become poor, and when it exceeds about 5, the skin adhesion during application and use may be drastically degraded due to skin perspiration that occurs under and/or around the system during use.

In some embodiments, the proportion of the organic liquid, including the permeation enhancer, relative to the total weight of the adhesive layer is not more than about 20%. When the proportion of the organic liquid exceeds about 20%, the cohesion of the adhesive layer drastically decreases and cohesive failure tends to occur easily.

E. Optional Additives

In some embodiments, the adhesive layer may contain, as optional components, other additives. Examples of optional additives include (1) esters, such as glycerine fatty acid ester, sorbitan fatty acid ester, etc., (2) organic solvents having a high boiling point, such as dimethyl sulfoxide, N-methylpyrrolidone etc., and (3) absorption promoters, such as pyrrolidonecarboxylate etc., and the like. Those skilled in the art can select suitable additives guided by the principles of maintaining the effects of the transdermal composition, e.g., suitable drug delivery and adhesive properties. In some embodiments, the proportion of additives as optional components is not more than about 15% of the total weight of the adhesive layer, such as not more than 15%.

The adhesive layer may be of any suitable thickness. In some embodiments, the thickness of the adhesive layer is generally about 30 μm to about 300 μm, and including about 60 μm to about 180 μm. In one embodiment, the thickness of the adhesive layer is 30 μm to 300 μm, including 60 μm to 180 μm.

2. Support

As noted above, the transdermal compositions described herein comprise a support. The support used is not particularly limited, and suitable supports for transdermal compositions are known in the art.

In some embodiments, the support is substantially impermeable to the drug (Sufentanil) and other components of the preparation. Thus, for example, in some embodiments the composition does not suffer from a decrease in the preparation content due to the loss of Sufentanil, additives, and the like from the adhesive layer through leakage via the support. Exemplary supports include, but are not limited to, (1) single films of polyester, nylon, Saran®, polyethylene, polypropylene, polyvinyl chloride, ethylene-ethyl acrylate copolymer, polytetrafluoroethylene, Surlyn®, metal foil, and the like, or (2) lamination films of these and the like can be used.

In some embodiments, the support is a lamination film of a non-porous plastic film made from the above-mentioned material and a porous film, so as to improve the adhesive force (anchor property) between the support and an adhesive layer. In this case, the adhesive layer may be formed on the porous film side. As such a porous film, one capable of improving the anchor property with the adhesive layer can be employed. Specifically, paper, woven fabric, non-woven fabric, knitted fabric, mechanically perforated sheet, and the like can be used. In some embodiments, which may have advantageous handling properties and the like, the support comprises paper, woven fabric, and/or non-woven fabric.

The porous film may be of any suitable thickness. In some embodiments, the porous film has a thickness of about 10 μm to about 200 μm, including 10 μm to 200 μm. Such embodiments may exhibit improved anchor property, good flexibility of adhesive preparation as a whole, and good adhesion operability and the like. In the case of a thin preparation such as a plaster type preparation or a pressure-sensitive adhesive type preparation, a porous film having a thickness of about 10 μm to about 100 μm, including 10 μm to 100 μm, can be employed.

When a woven fabric or a non-woven fabric is used as the porous film, the fabric weight may be about 5 g/m² to about 30 g/m², and including about 6 g/m² to about 15 g/m². In one embodiment, the fabric weight is 5 g/m² to 30 g/m², including 6 g/m² to 15 g/m².

In some embodiments, the preferred support is a lamination film of a polyester film (such as polyethylene terephthalate film) having a thickness of about 1.5 μm to about 6 μm, including 1.5 μm to 6 μm, and a non-woven polyester (such as polyethylene terephthalate) fabric having a fabric weight of about 6 g/m² to about 12 g/m², including 6 g/m² to 12 g/m². It has been found that a support comprising this type of film, in combination with the adhesive layer formulation described above, provides for several advantages. For example, the support material provides a flexibility of such an appropriate degree that the composition can follow the curve and movement of the skin surface without generating a significant sense of incongruity when applied to the skin surface.

In some embodiments, the support is translucent or transparent, allowing for a substantially clear, transparent or translucent transdermal composition. These embodiments may offer advantages of improved aesthetic characteristics of the system.

In some embodiments, the support is printed, while in other embodiments the support is unprinted.

These properties are highly desirable based upon patient compliance and patient preference criteria.

3. Membrane

In some embodiments, the percutaneously absorbable adhesive preparations or transdermal compositions described herein comprise a drug-release control membrane to control, e.g., prolong, drug delivery. The drug-release control membrane (e.g., rate controlling membrane) is not particularly limited, and suitable membranes are known in the art that control drug release from the percutaneously absorbable adhesive preparations (transdermal compositions). Exemplary drug-release control membranes include, but are not limited to, ethylene vinyl acetate films, microporous films, and the like. An exemplary ethylene vinyl acetate film used as a rate controlling membrane can comprise vinyl acetate in a concentration of about 2% to about 30%, including about 4.5% to about 19%, by weight of the membrane. In one embodiment, the vinyl acetate concentration is 2% to 30%, including 4.5% to 19%, by weight of the membrane. An exemplary microporous film used as a rate controlling membrane in the preparation of the invention can have a pore diameter of about 0.01 μm to about 1 μm, including 0.01 μm to 1 μm, and can be made from compounds such as polyester, nylon, polyethylene, polypropylene, polyvinyl chloride, ethylene-ethyl acrylate copolymer, polytetrafluoroethylene, and the like. The thickness of the drug-release control membrane (also known as a rate controlling membrane) can be any suitable thickness, and is generally about 10 μm to about 200 μm, including about 20 μm to about 100 μm. In one embodiment, the thickness of the membrane is 10 μm to 200 μm, including 20 μm to 100 μm.

4. Release Liner

The percutaneously absorbable adhesive preparations, or transdermal compositions, described herein may have a release liner laminated thereon to protect the adhesive surface of the adhesive layer until use. The release liner is not particularly limited as long as it can be subjected to a peel treatment and has a sufficient peelability; such release liners are known in the art. Examples thereof include, but are not limited to, films of polyester, polyvinyl chloride, polyvinylidene chloride, polyethylene terephthalate and the like, paper such as quality paper, glassine and the like, a laminate film of quality paper, glassine etc. with polyolefin, and the like, which have been subjected to a peel treatment comprising applying silicone resin, fluororesin, and the like to the surface to be in contact with the adhesive layer. The thickness of the release liner can be any suitable thickness, and is generally about 10 μm to about 200 μm, including about 25 μm to about 100 μm. In one embodiment, the release liner has a thickness of 10 μm to 200 μm, including 25 μm to 100 μm.

In some embodiments, the release liner is made from a polyester (such as polyethylene terephthalate) resin, which may offer advantageous barrier properties and cost advantages. In specific embodiments, such a release liner has a thickness of about 25 μm to about 100 μm, including 25 μm to 100 μm.

5. Packaging

In some embodiments, the percutaneously absorbable adhesive preparation, or transdermal composition, is sealed in packaging material until before use for preservation, transportation, and the like. The packaging may take any suitable form and be accomplished by any suitable means, and suitable packaging materials and methods are known in the art. The packaging method includes, for example, superimposing one sheet or several sheets of the percutaneously absorbable adhesive preparation, packing them with a packaging material, and heat-sealing the part surrounding the preparations. Thus, for example, a transdermal composition can be placed between two layers of packaging material, and one or more edges of the packaging material can be heat sealed. Other packaging materials and methods also can be used.

The packaging material is not particularly limited and may be, for example, a sheet or a film. In some embodiments, which may have advantageous packaging and air tightness, the packaging material is heat-sealable. Specific examples of suitable packaging materials include those using a plastic sheet having heat sealability, such as polyethylene, Surlyn®, ethylene-vinyl acetate copolymer, ethylene-vinyl alcohol copolymer, polyacrylonitrile copolymer, polyvinyl alcohol copolymer and the like. A laminate of a gas impermeable film such as polyester film, metal foil, and the like may be used to prevent volatilization, scattering, and the like of Sufentanil, which is an active ingredient, present in the adhesive preparation.

The packaging material may have any suitable thickness, and packaging materials having a thickness of generally about 10 μm to about 200 μm, including 10 μm to 200 μm, are typically used. In one embodiment, the above-mentioned packaging material comprising a polyacrylonitrile copolymer having high barrier property in the innermost layer is used. In some embodiments, design strategies are used to prevent degradation of handling property (e.g., easy removal from a package) such as may be caused by the bleeding of the adhesive component and the like. Exemplary designs include an emboss processing of the packaging material, a dry etching processing to slightly enlarge the aforementioned liner part (e.g., innermost layer) as compared to the preparation, a package formed by blister molding to reduce the contact area, and the like. Such packaging designs are known in the art. In a typical method of use, the percutaneously absorbable adhesive preparation, or transdermal composition, is removed from any packaging inmmediately before use, any release liner is removed, and the (exposed) adhesive surface is adhered to the skin surface.

Methods of Manufacture

The percutaneously absorbable adhesive preparations or transdermal compositions described herein can be produced by any means known in the art. For example, the two kinds of polyisobutylene having different viscosity average molecular weights, tackifier, permeation enhancer, any other optional additives, and Sufentanil can be dissolved in a suitable solvent, such as toluene etc., to obtain an adhesive solution. The adhesive solution can be applied to a release liner, and then dried to form an adhesive layer. The adhesive layer can then be laminated to a support. Alternatively, the above-mentioned adhesive solution can be applies to a support and dried to form an adhesive layer. If the adhesive solution is applied thickly, the solution may not be dried uniformly. Thus, to avoid this problem and ensure a certain desired thickness of the adhesive layer, the adhesive solution may be applied in thin layers two or more times, until the desired thickness is reached. The layers may optionally be dried or permitted to dry between applications.

Methods of Treatment Using the Sufentanil Preparations of the Invention

The percutaneously absorbable adhesive preparations, or transdermal compositions, of the invention are useful in treating subjects in need of narcotic pain relief. In an exemplary method of treatment a Sufentanil preparation or transdermal composition according to the invention is applied to the skin of the subject, whereby delivery of a therapeutically effective amount of Sufentanil is achieved. The Sufentanil preparations (e.g., transdermal compositions) described herein may be particularly useful in managing persistent, moderate to severe chronic pain that requires continuous, around-the-clock opioid administration for an extended period of time, and that cannot be managed by other means, such as by non-steroidal analgesics, opioid combination products, or immediate release opioids.

In some embodiments, the method includes applying a transdermal composition, for example, about 1, about 2, or about 3 times per week, including 1, 2, or 3 times per week. Thus, for example, a first composition can be applied and, from about 1 to about 3 days later or longer, a second composition can be applied. In some embodiments, the first composition is removed before the second composition is applied, or is removed substantially simultaneously with application of the second composition. In some embodiments, additional Sufentanil transdermal compositions are applied 1 to 6 times, or as many times as needed, after the initial administration, with or without the removal of the original and subsequent compositions.

In some embodiments, the Sufentanil preparation or transdermal provides therapeutic levels of Sufentanil for about 1 day, about 2 days, about 3 days, about 4 days, about 5 days, about 6 days, or about up to one week (e.g., about 7 days), or longer, including for 1, 2, 3, 4, 5, 6, or 7 days. Thus, for example, a composition may comprise an amount of Sufentanil effective to provide therapeutic levels of Sufentanil in the subject for a period of time of about 1 day, about 2 days, about 3 days, about 4 days, about 5 days, about 6 days, and about one week, or longer, including including 1, 2, 3, 4, 5, 6, or 7 days, or longer. As noted above, a new composition may be applied about 1 to about 3 times, or about seven times, per seven days (e.g., a new composition may be applied about 1, 2, or 3 times a week, or daily), as needed, for as long as needed for treatment.

In some embodiments the Sufentanil transdermal compositions comprise an amount of Sufentanil effective to deliver from about 0.5, about 1, about 1.5 or to about 2 μg/hr of Sufentanil per cm² of the surface area of the composition to the subject, including 0.5, 1, 1.5 or 2 μg/hr of Sufentanil per cm². In particular embodiments, the transdermal composition may comprise an amount of Sufentanil effective to maintain delivery of Sufentanil at about 0.5, about 1, about 1.5 or about 2 μg/hr per cm² of the surface area of the composition for the duration of the application, e.g., for from about 1 day to up to about one week, or longer. In one embodiment, the transdermal composition comprises an amount of Sufentanil effective to maintain delivery of Sufentanil at 0.5, 1, 1.5 or 2 μg/hr per cm² of the surface area of the composition for the duration of the application, e.g., for from 1 day to one week, or longer. The transdermal composition may comprise an amount of Sufentanil effective to deliver greater than 1 μg/hr Sufentanil per cm² of the surface area of the composition in the first 24 hours after initial application, and/or an amount effective to achieve, after the first 24 hours after initial application, a plasma concentration of Sufentanil in the subject of at least 1 pg/ml per cm² of the surface area of the composition.

As noted above, it has been found that, after application of the transdermal compositions described herein to human subjects, Sufentanil is not detected in the blood, for about 30 minutes to about two hours after initial application, and that Sufentanil may not be detected in the blood for up to about six hours after initial application. Thus, for example, the plasma concentration of Sufentanil in a subject may reach greater than about 2 pg/ml, such as greater than 2 pg/ml, within six hours after initial application of the Sufentanil transdermal composition. It also has been found that peak plasma concentrations (Cₘₐₓ) may be achieved within from about 12 to about 72 hours (Tₘₐₓ), including about 24 hours, after application of the transdermal compositions described herein.

In some embodiments, the transdermal compositions of the present invention achieve peak plasma concentrations of Sufentanil in the subject of from about 1 to about 30 pg/ml per cm² of the surface area of the composition, including about 1, about 5, about 10, about 15, about 20, about 25, or about 30 pg/ml per cm² of the surface area of the composition, including 1, 5, 10, 15, 20, 25 or 30 pg/ml per cm². In one embodiment, the transdermal composition comprises an amount of Sufentanil effective to achieve a peak plasma concentration of Sufentanil in the subject of about 30 to about 90 pg/ml, including about 30, about 35, about 40, about 45, about 50, about 55, about 60, about 65, about 70, about 75, about 80, about 85 or about 90 pg/ml, including 30, 35, 40, 45, 50, 55, 60, 65, 70, 75, 80, 85 or 90 pg/ml. In one particular aspect of this embodiment, the composition has a surface area of about 3 cm², including about 2.7 cm², including 2.7 cm².

Thus, for example, the compositions may comprise an amount of Sufentanil effective to achieve the above-described peak plasma concentrations of Sufentanil in the subject. In a specific embodiment, the peak plasma concentration in the subject is achieved at about 12 to about 72 hours, including about 24 hours, after initial application of the Sufentanil transdermal composition, including 12 to 72, or 24, hours after initial application.

The following examples are given to illustrate the present invention. It should be understood, however, that the spirit and scope of the invention is not to be limited to the specific conditions or details described in these examples but should only be limited by the scope of the claims that follow.

All references identified herein, including U.S. patents, are hereby expressly incorporated by reference in their entirety.

### EXAMPLES

The present invention is explained in detail in the following by referring to Examples, which are not to be construed as limiting. In the following, “part” and “%” mean “parts by weight” and “wt %”, respectively.

### Example and Comparative Example

### Manufacture of Samples

The compositions having mixing ratios of Samples Nos. 1-23 shown in the following Table 3, Table 7, and Table 8 were dissolved in toluene to give an adhesive layer coating solution having a solute concentration of 30%. This solution was applied to a polyethylene terephthalate (PET) liner after a silicone peel treatment to provide a weight of adhesive layer after drying as indicated in the Tables. This was dried in a hot-air circulation oven at 105° C. for 5 min to provide an adhesive layer.

A non-woven fabric surface of a support made of a 2 μm thick PET film and a PET non-woven fabric (12 g/m²) adhered to each other with a polyester adhesive was laminated on the adhesive layer, and aged at room temperature for 2 days to give a sheet-like percutaneously absorbable adhesive preparation, e.g., transdermal composition. In the Experimental Examples, Samples Nos. 1 and 17 are Comparative Examples.

The compositions comprising Sample Nos. 24-29 and described in Experimental Example 5, and Table 9 are multi-layer systems comprising a rate-controlling membrane. These compositions were prepared by preparing the adhesive layer in two parts. A reservoir layer comprising an adhesive layer comprising Sufentanil as described above was produced in the manner described above. A contact adhesive layer was made by coating a solvated polyisobutylene composition onto a release film, drying the film, and subsequently laminating the coated release film to a rate-controlling membrane. The reservoir layer and the contact adhesive layer were combined by removing the release liner of the reservoir layer and laminating the reservoir layer to the membrane side of the contact adhesive layer.

In this example, the contact adhesive layer was prepared as an inactive adhesive layer, but this layer optionally can be prepared as including Sufentanil.

### Experimental Example 1 (Solubility of Sufentanil in Additive)

Sufentanil (0.1 g) was measured, each of the additives (1 g) shown in Table 1 was added, and the mixture was stirred at room temperature (around 25° C.) for 30 min. The solutions were filtered, the filtrate (0.1 g) was precisely measured, dissolved in methanol, and the amount of Sufentanil dissolved therein was measured by HPLC.

Table 1 shows the solubility of Sufentanil in each additive. Based on this data, isopropyl myristate (IPM: NIKKO CHEMICALS IPM-100) and octyldodecanol (ODO: Cognis Co. EUTANOL® G) were considered to be suitable for transdermal compositions and were used to perform the following tests.

TABLE 1
Solubility of Sufentanil in additive (permeation enhancer)

| | | Solubility |
|---|---|---|
| No. | Additive | (mg/g)* |
| 1 | Ethyl oleate | 44.8 |
| 2 | Isopropyl myristate | 44.9 |
| 3 | Isopropyl palmitate | 36.7 |
| 4 | Isostearyl alcohol | 65.5 |
| 5 | Octyldodecanol | 60.2 |
| 6 | Polysorbate 80 | 36.9 |
| 7 | Sorbitan trioleate | 103.8 |

*Solubility is the amount of Sufentanil that is dissolved in 1 g of additive (mg)

### Experimental Example 2 (Permeation Test of Skin Removed from Mice, Analysis of Maximum Permeation Rate, and Evaluation of Appearance after a 3 Month Time Period)

1. Permeation Test of the Skin Removed from Mouse

Percutaneously absorbable adhesive preparations having the composition shown in Sample Nos. 1-18 of Table 3 (various combinations of factor and level shown in Table 2), which were produced by the above-mentioned method, were subjected to a permeation test according to the following method using the skin removed from mouse. For the test, a cell permeation test apparatus was used.

Each preparation was punched out two at a time in φ6 mm to provide samples. The skin was removed from 8-week-old (male) mice. As the receptor solution, 0.15 mol/L citric acid-phosphate buffer was used.

One sheet of preparation punched out in φ6 mm (0.2826 cm²) was adhered to the removed skin. The removed skin was set in a diffusion cell by being placed therein with the surface having the adhered preparation facing upward. A receptor solution was fed at about 2.5 ml/hr, and the receptor solution discharged from the diffusion cell was recovered in vial containers as fractions at 4, 5, 6, 7, 8, 9, 12, 15, 18, 21 and 24 hr. After recovery, the weight was accurately measured. The Sufentanil concentration of the recovered solution was measured by HPLC, and the amount of permeation and permeation rate were calculated.

The following “Predicted Analysis by L18 experimental design” was first conducted with Fentanyl, as described in U.S. patent application Ser. No. 11/196,935, filed on Aug. 4, 2005, which is specifically incorporated by reference. As noted above, Sufentanil is a Fentanyl analogue. Based on the relationship between Fentanyl and Sufentanil, it is anticipated that the results previously obtained with Fentanyl in a L18 experimental design would be expected to be very similar, or identical, to those predicted to be obtained for Sufentanil.

2. Predicted Analysis by L18 Experimental Design

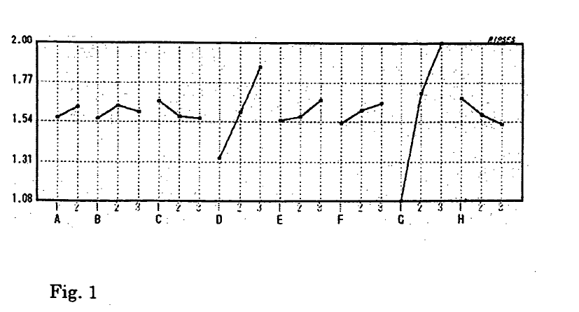

The maximum permeation rate of Sufentanil could be applied to a factor effect analysis based on the larger-the-better characteristic according to L18 experimental design. Table 2 shows factors (e.g., mixing ratio, molecular weight, concentration) and levels, Table 3 shows eighteen (18) proposed samples based on combinations according to the L18 orthogonal table, Table 4 shows the predicted maximum permeation speed in each sample, Table 5 shows predicted variance analysis results (which are expected to demonstrate that significant factors are D (isopropyl myristate concentration as a percent of the adhesive layer) and G (Sufentanil concentration as a percent of the adhesive layer)), Table 6 shows the step average, and FIG. 1 shows a schematic factor effect of Table 6 in the form of a diagram. In Table 5, factors A-G are controlling factors A-G in Table 2, H and e are errors irrelevant to each factor, T is diffusion of the whole, f is degree of freedom, S is sum of squares, and V is diffusion. In Table 6, factors A-G are controlling factors A-G in Table 3, H is an error irrelevant to the factor, levels 1-3 are levels 1-3 of Table 2, and the numerical values in the Table show SN ratio based on larger-the-better characteristic at each level. The SN ratio based on larger-the-better characteristic was determined by the formula:
SN ratio=−10×Log(1/y²)(db) wherein y is a characteristic value.

The estimated process average in FIG. 1 is an estimated value of the effect expected to be achieved by the combination of most effective levels. In FIG. 1, the underlined levels were employed to obtain the estimated value of process average.

From the predicted results, the skin permeability of the Sufentanil in the percutaneously absorbable adhesive preparation or transdermal compositions described herein is controlled by changing the ratio of two kinds of polyisobutylene having different molecular weights, a tackifier, and a permeation enhancer which achieves various levels of skin permeability. When compared to the error (row H), Sufentanil concentration and IPM concentration are expected to have significant effects on the skin permeability.

The details of the analysis method according to the L18 experimental design follow “Quality Engineering Course 4 Experiments for Quality Design, Genichi Taguchi, Japanese Standards Association”. For the analysis, an analysis software, “RIPSES for windows” RICOH RIPSES DEVELOPMENT G CO., Ltd., was used.
The abbreviations in Table 2 and Table 3 have the following meanings:
B80: Oppanol® B80 (BASF) polyisobutylene, viscosity average molecular weight 820,000
B100: Oppanol® B100 (BASF) polyisobutylene, viscosity average molecular weight 1,110,000
P100: ARKON® P100 (Arakawa Chemical Industries, Ltd.) tackifier, alicyclic saturated hydrocarbon resin, average molecular weight 610
B12: Oppanol® B12 (BASF) polyisobutylene, viscosity average molecular weight 55,000
IPM: IPM-100 (NIKKO Chemicals) isopropyl myristate
ISO: (Wako Pure Chemical Industries, Ltd.) reagent isostearyl alcohol

SUF: Sufentanil

TABLE 2
Factors and levels

| | | | | | E | | |
|---|---|---|---|---|---|---|---|
| | | B | | D | Low | F | G |
| | A | High | | IPM | molecular | ISO | SUF |
| | High | molecular | C | Conc. (%)/ | weight | Conc. (%)/ | Conc. (%)/ |
| | molecular | weight | P100 | whole | rubber | whole | whole |
| Control | weight | rubber | mixing | adhesive | B12 | adhesive | adhesive |
| factor | rubber | Mixing ratio | ratio | layer | Mixing ratio | layer | layer |
| Level 1 | B80 | 25 | 17 | 0 | 42 | 0 | 1.5 |
| Level 2 | B100 | 30 | 25 | 3 | 50 | 3 | 2 |
| Level 3 | — | 35 | 33 | 5 | 58 | 5 | 2.5 |

In Table 2, B, C and E show mixing ratios, and D, E and F show (%) relative to the total weight of the adhesive layer.

TABLE 3

| Sam- | | | | | | | | |
|---|---|---|---|---|---|---|---|---|
| ple | | | | | | | | weight of |
| No. | SUF | B80 | B100 | B12 | P100 | IPM | ISO | adhesive layer |
| 1 | 1.5 | 29.5 | — | 49.3 | 19.9 | 0.0 | 0.0 | 0.167 g/10 cm² |
| 2 | 2.0 | 23.0 | — | 46.0 | 23.0 | 3.0 | 3.0 | 0.125 g/10 cm² |
| 3 | 2.5 | 18.9 | — | 43.8 | 24.9 | 5.0 | 5.0 | 0.100 g/10 cm² |
| 4 | 2.5 | 29.2 | — | 48.7 | 16.6 | 0.0 | 3.0 | 0.100 g/10 cm² |
| 5 | 1.5 | 24.0 | — | 46.5 | 20.0 | 3.0 | 5.0 | 0.167 g/10 cm² |
| 6 | 2.0 | 26.6 | — | 37.2 | 29.2 | 5.0 | 0.0 | 0.125 g/10 cm² |
| 7 | 2.0 | 33.5 | — | 40.2 | 16.3 | 3.0 | 5.0 | 0.125 g/10 cm² |
| 8 | 2.5 | 29.4 | — | 42.0 | 21.0 | 5.0 | 0.0 | 0.100 g/10 cm² |
| 9 | 1.5 | 26.5 | — | 44.0 | 25.0 | 0.0 | 3.0 | 0.167 g/10 cm² |
| 10 | 2.0 | — | 22.5 | 52.2 | 15.3 | 5.0 | 3.0 | 0.125 g/10 cm² |
| 11 | 2.5 | — | 25.1 | 42.2 | 25.1 | 0.0 | 5.0 | 0.100 g/10 cm² |
| 12 | 1.5 | — | 22.1 | 44.2 | 29.2 | 3.0 | 0.0 | 0.167 g/10 cm² |
| 13 | 2.5 | — | 27.0 | 52.2 | 15.3 | 3.0 | 0.0 | 0.100 g/10 cm² |
| 14 | 1.5 | — | 28.0 | 39.2 | 23.3 | 5.0 | 3.0 | 0.167 g/10 cm² |
| 15 | 2.0 | — | 24.7 | 41.2 | 27.2 | 0.0 | 5.0 | 0.125 g/10 cm² |
| 16 | 1.5 | — | 30.4 | 43.4 | 14.8 | 5.0 | 5.0 | 0.167 g/10 cm² |
| 17 | 2.0 | — | 29.1 | 48.2 | 20.8 | 0.0 | 0.0 | 0.125 g/10 cm² |
| 18 | 2.5 | — | 29.1 | 34.9 | 27.5 | 3.0 | 3.0 | 0.100 g/10 cm² |

In Table 3, each numerical value shows mixing wt %.

TABLE 4
Maximum permeation rate (n = 2 average) and evaluation
of appearance after a Three (3) month time period

| Sample | Maximum permeation rate |
|---|---|
| No. | (μg/cm²/h) |
| 1 | 0.83 |
| 2 | 1.64 |
| 3 | 2.26 |
| 4 | 1.72 |
| 5 | 1.27 |
| 6 | 1.81 |
| 7 | 1.63 |
| 8 | 2.15 |
| 9 | 0.77 |
| 10 | 2.14 |
| 11 | 1.65 |
| 12 | 0.83 |
| 13 | 2.13 |
| 14 | 1.28 |
| 15 | 1.59 |
| 16 | 1.50 |
| 17 | 1.44 |
| 18 | 2.09 |

TABLE 5

| Factor | f | S | V |
|---|---|---|---|
| A | 1 | 0.0181 | 0.0181 |
| B | 2 | 0.0169 | 0.0084 |
| C | 2 | 0.0354 | 0.0177 |
| D | 2 | 0.8217 | 0.4108 |
| E | 2 | 0.0486 | 0.0243 |
| F | 2 | 0.0430 | 0.0215 |
| G | 2 | 2.6525 | 1.3263 |
| H | 2 | 0.0705 | 0.0352 |
| e | 2 | 0.0290 | 0.0145 |
| T | 17 | 3.7356 | — |

TABLE 6

| | | | | Total |
|---|---|---|---|---|
| Factor | Level 1 | Level 2 | Level 3 | average |
| A | 1.5644 | 1.6278 | — | 1.5961 |
| B | 1.5583 | 1.6333 | 1.5967 | |
| C | 1.6583 | 1.5717 | 1.5583 | |
| D | 1.3333 | 1.5983 | 1.8567 | |
| E | 1.5483 | 1.5717 | 1.6683 | |
| F | 1.5317 | 1.6067 | 1.6500 | |
| G | 1.0800 | 1.7083 | 2.0000 | |
| H | 1.6783 | 1.5833 | 1.5267 | |

### Experimental Example 3 (In Vitro Permeation Test in the Human Epidermis Obtained from Cryopreserved Cadaver Skin)

1. Permeation Test of Human Epidermis Obtained from Cryopreserved Cadaver Skin

Percutaneously absorbable adhesive preparations (transdermal compositions) having the compositions of Sample Nos. 19-22 were produced by the above-mentioned method and subjected to a permeation test using human epidermis obtained from cryopreserved cadaver skin. For the test, a cell permeation test apparatus was used.

The preparations were punched out (five at a time) in φ7.938 mm (preparation application area: 0.4946 cm²) to provide samples. The human epidermis was obtained from cryopreserved cadaver skin which was then separated to isolate the epidermis layers using the heat separation method. As the receptor solution, 0.9% Sodium chloride containing 0.01% Sodium azide was used.

(Permeation Test Method)

One sheet of preparation punched out in φ7.938 mm (0.4946 cm²) was adhered to human epidermis obtained from cryopreserved cadaver skin. The epidermis was set in a diffusion cell by being placed therein with the surface having the adhered preparation facing upward. A certain amount of the receptor solution was sampled with the lapse of time, the concentration of Sufentanil in the receptor solution was measured by HPLC, and the flux was calculated.

The abbreviations in Table 7 have the following meanings:
H300: PANALENE® H300 (BP Amoco Chemical Co.) polybutene, number average molecular weight (GPC)1300
ODO: EUTANOL® G NF (Cognis Co.) octyldodecanol

SUF, B80, B12, and IPM are similar to those shown in Table 2 and Table 3.

TABLE 7

| | | | | | | | weight of |
|---|---|---|---|---|---|---|---|
| | | | | | | | adhesive |
| Sample No. | SUF | B80 | B12 | H300 | IPM | ODO | layer |
| 19 | 1.7 | 26.0 | 39.0 | 23.3 | 3.0 | 7.0 | 15 mg/cm² |
| 20 | 1.7 | 24.5 | 36.8 | 22.0 | 10.0 | 5.0 | 15 mg/cm² |
| 21 | 2.5 | 24.9 | 37.3 | 22.3 | 3.0 | 10.0 | 15 mg/cm² |
| 22 | 4.0 | 24.4 | 36.6 | 22.0 | 3.0 | 10.0 | 15 mg/cm² |

In Table 7, each numerical value shows mixing wt%.

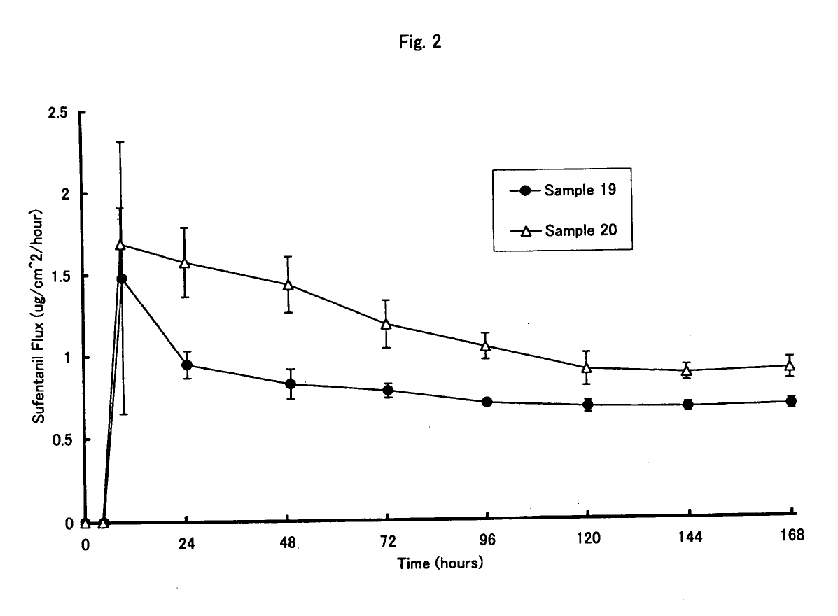

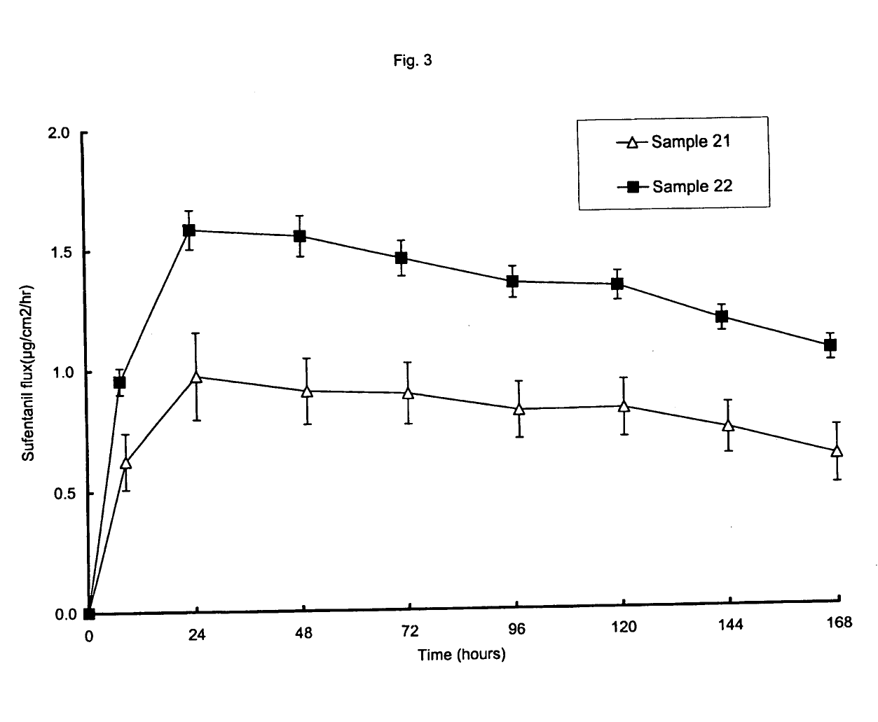

The results of in vitro percutaneous flux of Sufentanil through human epidermis are shown in FIG. 2 and 3. From these results, the percutaneously absorbable adhesive preparation described herein was confirmed to exhibit effective transdermal absorbability, and the transdermal absorption lasted for 1 week.

### Experimental Example 4 (Clinical Study)

1. Clinical Study

A percutaneously absorbable adhesive preparation (transdermal composition) having the compositions of Sample No. 23 was produced by the above-mentioned method and subjected to a permeation test in eight (8) human subjects.

The preparation was punched out in 2.7 cm² to provide a sample. One sheet of preparation punched out in 2.7 cm² was applied to human subjects. Blood samples were taken over time, and the blood concentration of Sufentanil was measured by HPLC/MS/MS.

SUF, B80, B12 and IPM are similar to those shown in Table 2 and Table 3. H300 and ODO are similar to those shown in Table 7.

TABLE 8

| | | | | | | | Weight of |
|---|---|---|---|---|---|---|---|
| Sample No. | SUF | B80 | B12 | 300 | IPM | ODO | adhesive layer |
| 23 | 2.5 | 24.9 | 37.3 | 22.3 | 3.0 | 10.0 | 10 mg/cm² |

In Table 8, each numerical value shows mixing wt%.

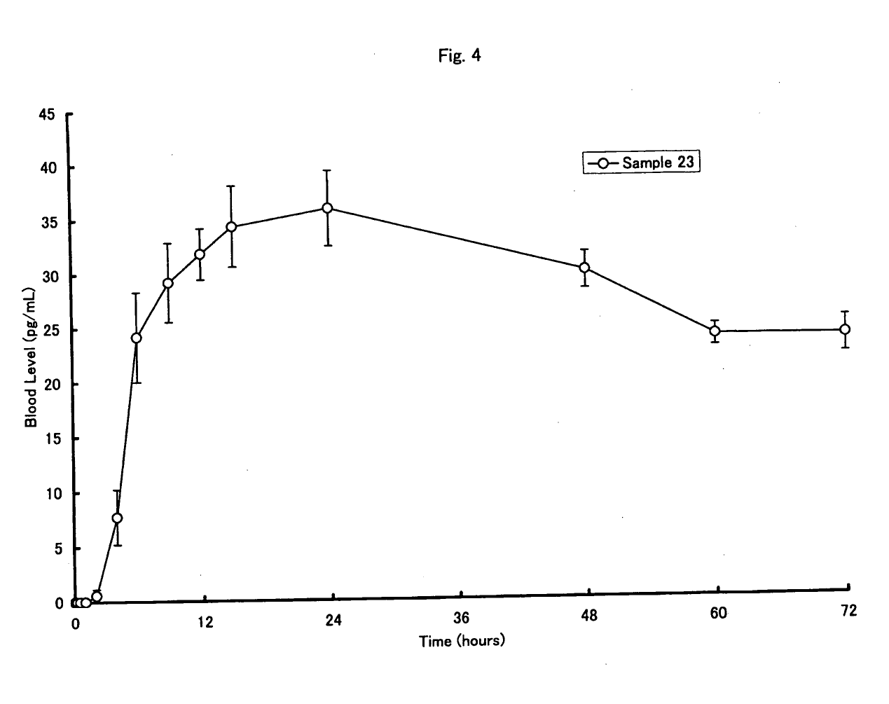

The results are shown in FIG. 4. From these results, the percutaneously absorbable adhesive preparations described herein was confirmed to exhibit sufficient blood levels while the preparations were applied. No skin initation or other skin side effects associated with the Sufentanil preparations were observed.

### Experimental Example 5 (In Vitro Permeation Test in the Human Epidermis Obtained from Cryopreserved Cadaver Skin)

1. Permeation Test of Human Epidermis Obtained from Cryopreserved Cadaver Skin

Percutaneously absorbable adhesive preparations (transdermal compositions) having the compositions of Sample Nos. 24-29 were produced by the above-mentioned method and subjected to a permeation test of the human epidermis obtained from cryopreserved cadaver skin. For the test, a cell permeation test apparatus was used.

The preparations were punched out (five at a time) in φ7.938 mm (preparation application area: 0.4946 cm²) to provide samples. The human epidermis was obtained from cryopreserved cadaver skin which was then separated to isolate the epidermis layers using the heat separation method. As the receptor solution, 0.9% Sodium chloride containing 0.01% Sodium azide was used.

(Permeation Test Method)

One sheet of preparation punched out in φ7.938 mm (0.4946 cm²) was adhered to human epidermis obtained from cryopreserved cadaver skin. The epidermis was set in a diffusion cell by being placed therein with the surface having the adhered preparation facing upward. A certain amount of the receptor solution was sampled with the lapse of time, the concentration of Sufentanil in the receptor solution was measured by HPLC, and the flux was calculated.

Table 9 shows the following.
CoTran®9702: (3M) Controlled caliper ethylene acetate membrane, Caliper 50.8 μm, 9% Vinyl acetate
CoTran®9707: (3M) Controlled caliper ethylene acetate membrane, Caliper 50.8 μm, 4.5% Vinyl acetate
Celgard®2400: (Celgard Inc.) Microporous polypropylene membrane, Caliper 25 μm
Contact Layer: Inactive adhesive layer

SUF, B80, B12 and IPM are similar to those shown in Table 2 and Table 3. H300 and ODO are similar to those shown in Table 7.

TABLE 9

| | | | | | | | | | Contact |
|---|---|---|---|---|---|---|---|---|---|
| Sample | Reservoir Layer | | | | | | | Control | Layer |
| No | SUF | B80 | B12 | H300 | IPM | ODO | Coat Wt. | Membrane | Coat Wt. |
| 24 | 2.5 | 24.9 | 37.3 | 22.3 | 3.0 | 10.0 | 10 | CoTran ® 9702 | 3 |
| 25 | 3.3 | 24.6 | 36.9 | 22.2 | 3.0 | 10.0 | 10 | CoTran ® 9702 | 3 |
| 26 | 4.0 | 24.4 | 36.6 | 22.0 | 3.0 | 10.0 | 10 | CoTran ® 9702 | 3 |
| 27 | 5.0 | 24.1 | 36.2 | 21.7 | 3.0 | 10.0 | 10 | CoTran ® 9702 | 3 |
| 28 | 4.0 | 24.4 | 36.6 | 22.0 | 3.0 | 10.0 | 10 | CoTran ® 9707 | 3 |
| 29 | 4.0 | 24.4 | 36.6 | 22.0 | 3.0 | 10.0 | 10 | Celgard ® 2400 | 3 |

In Table 9, each numerical values of ingredients and coat weight show mixing wt % and mg/cm², respectively.

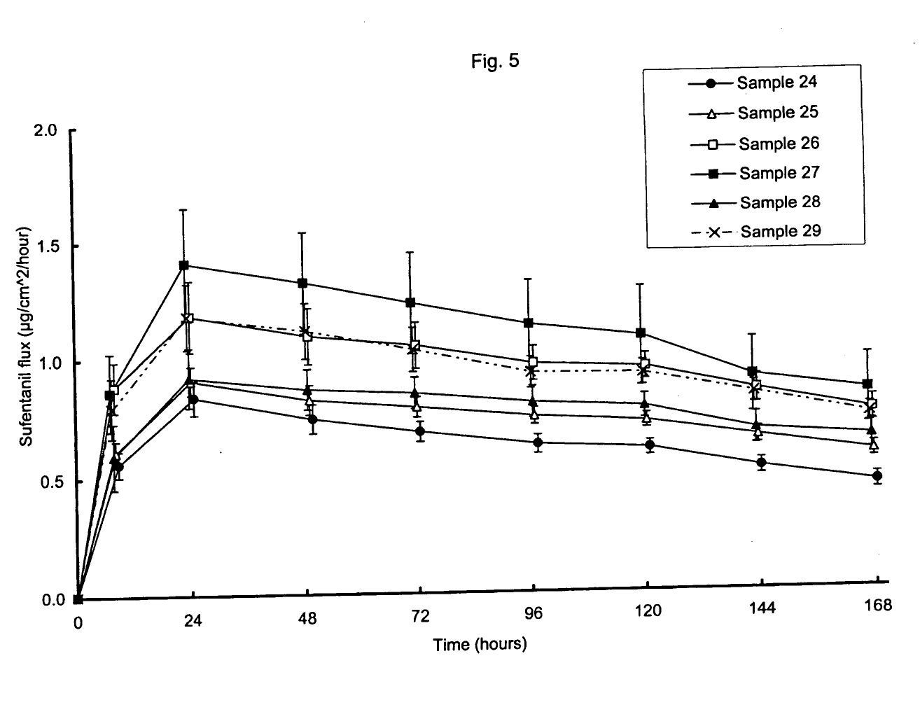

The results of in vitro percutaneous flux of Sufentanil through human epidermis are shown in FIG. 5. From these results, the percutaneously absorbable adhesive preparations described herein were confirmed to exhibit effective transdermal absorbability. The transdermal absorption lasted for 1 week.

### Experimental Example 6 (Clinical Study)

Clinical Study

A percutaneously absorbable adhesive preparation having the compositions of Sample No. 26 was produced by the above-mentioned method and subjected to a permeation test in eight (8) human subjects.

The preparation was punched out into 2.7 cm² compositions to provide a sample. One composition (comprising a sheet of preparation punched out into 2.7 cm²) was applied to each human subject. Blood samples were taken over time, and the blood concentration of Sufentanil was measured by HPLC/MS/MS. The threshold for detection was approximately 2 pg/ml.

SUF, B80, B12 and IPM are similar to those shown in Table 2 and Table 3. H300 and ODO are similar to those shown in Table 7.

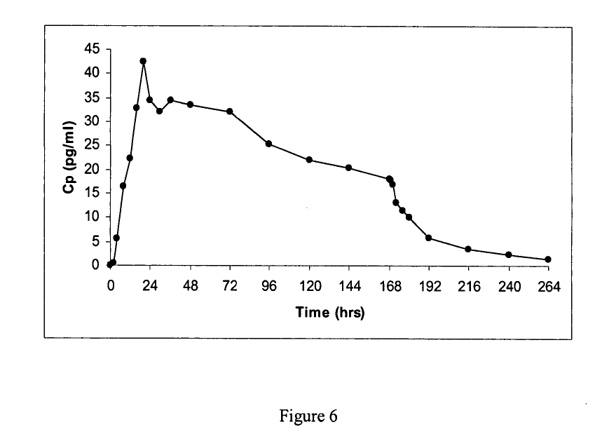

The results are shown in FIG. 6. From these results, the percutaneously absorbable adhesive preparations described herein were confirmed to exhibit sufficient blood levels while the preparations were applied. The incorporation of the controlling membrane provided for a more sustained and controlled delivery of Sufentanil compared to the system designed without the membrane. The observed skin irritation and other side effects associated with the Sufentanil preparation were minimal.

It will be apparent to those skilled in the art that various modifications and variations can be made in the methods and compositions of the present invention without departing from the spirit or scope of the invention. Thus, it is intended that the present invention cover the modifications and variations of this invention provided they come within the scope of the appended claims and their equivalents.

What is claimed is:

1. A transdermal composition comprising
(a) a support; and
(b) an adhesive layer laminated on one surface of the support,
wherein the adhesive layer comprises a subsaturated amount of Sufentanil, a polyisobutylene blend comprising a first polyisobutylene and a second polyisobutylene having different viscosity average molecular weights, a tackifier, and a permeation enhancer compatible with said polyisobutylene blend and said tackifier.

2. The composition of claim 1, wherein the Sufentanil comprises about 1.5% to about 5.0% of the total weight of said adhesive layer.

3. The composition of claim 1, wherein the first polyisobutylene has a viscosity average molecular weight of about 600000 to about 1500000 and the second polyisobutylene has a viscosity average molecular weight of about 40000 to about 85000.

4. The composition of claim 1, wherein the tackifier comprises a polybutene having a number average molecular weight of about 900 to about 2900.

5. The composition of claim 1, wherein the tackifier comprises an alicyclic saturated hydrocarbon resin having a number average molecular weight of about 500 to about 860.

6. The composition of claim 1, wherein the polyisobutylene blend comprises a mixing ratio of the first polyisobutylene:second polyisobutylene of about 1:1.2 to about 1:2, by weight, and wherein the second polyisobutylene has a smaller molecular weight than that of the first polyisobutylene.

7. The composition of claim 3, wherein the polyisobutylene blend comprises a mixing ratio of the first polyisobutylene:second polyisobutylene of about 1:1.3 to about 1:1.8, by weight, and wherein the second polyisobutylene has a smaller viscosity average molecular weight than that of the first polyisobutylene.

8. The composition of claim 1, wherein the tackifier comprises about 15% to about 30% of the total weight of said adhesive layer.

9. The composition of claim 1, wherein the permeation enhancer comprises not more than about 20% of the total weight of said adhesive layer.

10. The composition of claim 1, wherein the permeation enhancer is selected from the group consisting of a fatty acid alkyl ester and a branched long-chain alcohol.

11. The composition of claim 10, wherein the permeation enhancer comprises isopropyl myristate, isostearyl alcohol and/or octyldodecanol.

12. The composition of claim 10, wherein the permeation enhancer comprises a combination of a fatty acid alkyl ester and a branched long-chain alcohol, and a mixing ratio of the fatty acid alkyl ester branched long-chain alcohol is about 1:0.2 to about 1:5, by weight.

13. The composition of claim 11, wherein the permeation enhancer comprises a combination of a fatty acid alkyl ester and a branched long-chain alcohol, and a mixing ratio of the fatty acid alkyl ester:branched long-chain alcohol is about 1:0.3 to about 1:4 by weight.

14. The composition of claim 1, which comprises an amount of Sufentanil effective to provide therapeutic levels for a period of time selected from the group consisting of about 1 day, about 2 days, about 3 days, about 4 days, about 5 days, about 6 days, and about one week.

15. The composition of claim 1, which comprises an amount of Sufentanil effective to provide therapeutic levels of Sufentanil for up to about one week.

16. The composition of claim 1, comprising from about 0.5 mg to about 15 mg of Sufentanil.

17. The composition of claim 1, further comprising a rate controlling membrane.

18. The composition of claim 17, wherein the rate controlling membrane is an ethylene vinyl acetate film or a microporous film.

19. The composition of claim 18, wherein the ethylene vinyl acetate film comprises vinyl acetate in a concentration of about 2% to about 30%.

20. The composition of claim 18, wherein the rate controlling membrane is a microporous film having a pore diameter of about 0.01 μm to about 1 μm.

21. The composition of claim 17, wherein the rate controlling membrane comprises a compound selected from a group consisting of polyester, nylon, polyethylene, polypropylene, polyvinyl chloride, ethylene-ethyl acrylate copolymer, and polytetrafluoroethylene.

22. The composition of claim 18, wherein the thickness of the rate controlling membrane is about 10 μm to about 200 μm.

23. The composition of claim 22, wherein the thickness of the rate controlling membrane is about 20 μm to about 100 μm.

24. The composition of claim 1, wherein the support comprises a non-porous film laminated to a porous film.

25. The composition of claim 24, wherein the support consists of a non-porous film laminated to a porous film.

26. The composition of claim 1, wherein the support comprises woven fabric, nonwoven fabric, knitted fabric, paper, a mechanically perforated sheet, or a combination of two or more thereof.

27. The composition of claim 26, wherein the support consists of woven fabric, nonwoven fabric, knitted fabric, paper, a mechanically perforated sheet or a combination thereof

28. The composition of claim 26, wherein the support comprises woven or nonwoven fabric with a fabric weight of about 5 g/m² to about 30 g/m².

29. The composition of claim 24, wherein the support consists of s a polyester film and a nonwoven polyester fabric.

30. The composition of claim 1, wherein the support is translucent or transparent.

31. The composition of claim 1, wherein the Sufentanil transdermal composition has a size of about 1 cm² to about 30 cm².

32. The composition of claim 1, further comprising a release liner.

33. A method of treating a subject in need of pain relief, comprising applying to the skin of the subject a first Sufentanil transdermal composition comprising:
(a) a support; and
(b) an adhesive layer laminated on one surface of the support,
wherein the adhesive layer comprises a subsaturated amount of Sufentanil, a polyisobutylene blend comprising a first polyisobutylene and a second polyisobutylene having different viscosity average molecular weights, a tackifier, and a permeation enhancer compatible with said polyisobutylene blend and said tackifier.

34. The method of claim 33, wherein the Sufentanil transdermal composition comprises an amount of Sufentanil effective to provide therapeutic levels of Sufentanil in the subject for a period of time selected from the group consisting of about 1 day, about 2 days, about 3 days, about 4 days, about 5 days, about 6 days, and about one week.

35. The method of claim 33, wherein the Sufentanil transdermal composition comprises an amount of Sufentanil of from about 0.5 mg to about 15 mg.

36. The method of claim 33, further comprising applying to the skin of the subject a second Sufentanil transdermal composition comprising (a) a support; and (b) an adhesive layer laminated on one surface of the support, wherein the adhesive layer comprises a subsaturated amount of Sufentanil, a polyisobutylene blend comprising a first polyisobutylene and a second polyisobutylene having different molecular weights, a tackifier, and a permeation enhancer compatible with said two kinds of polyisobutylene and said tackifier.

37. The method of claim 36, further comprising removing the first Sufentanil transdermal composition from the skin of the subject prior to applying the second Sufentanil transdermal composition.

38. The method of claim 33, wherein the plasma concentration of Sufentanil in the subject remains less than about 2 pg/ml for a time period of about 30 minutes to about 2 hours after initial application of the Sufentanil transdermal composition.

39. The method of claim 33, wherein the method achieves a plasma concentration of Sufentanil in the subject of greater than about 2 pg/ml within six hours after initial application of the Sufentanil transdermal composition.

40. The method of claim 33, wherein the peak plasma concentration of Sufentanil in the subject is reached from about 12 hours to about 72 hours after initial application of the Sufentanil transdermal composition.

41. The method of claim 40, wherein the peak plasma concentration of Sufentanil in the subject is reached about 24 hours after initial application of the Sufentanil transdermal composition.

42. The method of claim 33, wherein the Sufentanil transdermal composition has a size of about 1 cm² to about 30 cm².

43. The method of claim 33, wherein the Sufentanil transdermal composition comprises an amount of Sufentanil effective to achieve a peak plasma concentration of Sufentanil in the subject of from about 1 to about 30 pg/ml per cm² of the composition.

44. The method of claim 43, wherein the peak plasma concentration in the subject is achieved at about 24 hours after initial application of the Sufentanil transdermal composition

45. The method of claim 33, wherein the Sufentanil transdermal composition comprises an amount of Sufentanil effective to deliver from about 0.5 to about 2 μg/hr Sufentanil per cm² of the composition over a duration of application of from at least about 1 day to about 7 days.

46. The method of claim 33, wherein the Sufentanil transdermal composition comprises an amount of Sufentanil effective to maintain a Sufentanil delivery rate of at least about 1 μg/hr Sufentanil per cm² of the composition for a duration of application of from at least about 1 day to about 7 days.

47. The method of claim 46, wherein the duration is a period of time selected from the group consisting of about 1 day, about 2 days, about 3 days, about 4 days, about 5 days, about 6 days, and about one week.

48. The method of claim 33, wherein the Sufentanil transdermal composition comprises an amount of Sufentanil effective to deliver at least 1 μg/hr Sufentanil per cm² of the composition during the first 24 hours after initial application.

49. The method of claim 48, wherein the Sufentanil transdermal composition comprises an amount of Sufentanil effective to achieve, after the first 24 hours after initial application, a plasma concentration of Sufentanil in the subject of at least 1 pg/ml per cm² of the composition.

50. The method of claim 33, wherein the Sufentanil transdermal composition comprises an amount of Sufentanil effective to achieve a peak plasma concentration of Sufentanil in the subject of about 30 to about 90 pg/ml.
